(19) United States
(12) Patent Application Publication
(10) Pub. No.: US 2026/0207524 A1
(43) Pub. Date: Jul. 23, 2026

(54) ACTIVE COMPOUND ENCAPSULATION SYSTEM AND METHOD OF FORMULATING THE SAME

(71) Applicant: Council for Scientific and Industrial Research, Pretoria (ZA)

(72) Inventors: Michel Lonji KALOMBO, Pretoria (ZA); Philip Wouter LABUSCHANGNE, Pretoria (ZA); Andri SWANEPOEL, Pretoria (ZA)

(21) Appl. No.: 19/139,701

(22) PCT Filed: Dec. 15, 2022
(86) PCT No.: PCT/IB2022/062274

§ 371 Date: Jun. 16, 2025

(51) Int. Cl. A61K 9/50; A61K 35/00; A61K 35/741

(52) U.S. Cl. CPC A61K 9/5089; A61K 9/5015; A61K 9/5026; A61K 35/741; A61K 2035/115

(57) ABSTRACT

An encapsulation system, for encapsulating at least one active compound as well as a method for formulating the same is described. The encapsulation system includes an encapsulating material and at least one active compound. The encapsulating material comprises a eutectic alternatively a non-eutectic (hypo or hyper eutectic) mixture and at least one additive. During the formulation of the encapsulation system the encapsulating material is homogenously combined with the at least one active compound in a solvent medium comprising a supercritical compound to form a matrix which encapsulates the active compound so that, in use, the active compound is protected from both ambient environmental factors and gastric attack. Protection against both ambient environmental factors and gastric attack leads to increased shelf and gastric stability which is desirable with respect to both the high costs associated with medicaments and specific release profiles for medicaments respectively.

### FIELD OF THE INVENTION

The invention relates to encapsulation systems for the encapsulation of labile active compounds (AC). More particularly, the invention relates encapsulation of the labile AC as a mechanism by which to protect the same from harsh surrounding environments both outside of and within the body. In particular, the invention relates to lipid-polymer hybrid encapsulation matrices and the method by which said matrices are formulated. The invention being predominantly utilised in pharmaceutical and/or nutraceutical applications. In such applications the invention is indicated for use in human and/or animal subjects (veterinary and/or agricultural) as medicaments or supplements.

### BACKGROUND TO THE INVENTION

Pharmaceutical compounds containing active pharmaceutical ingredients (APIs) and nutraceuticals are well known in the market. These drugs are used to treat various diseases in the case of pharmaceutical compounds and to provide various health benefits in the case of nutraceuticals. Many drugs contain natural compounds or derivatives thereof with one third of top selling pharmaceuticals being natural compounds, which are also widely used as nutraceuticals.

It is well known that the pharmaceutical industry is a multibillion-dollar industry with large amounts of investment going into research and development to develop new drugs, deliver known drugs more effectively and to provide for better shelf and gastric stability. Encapsulation, which is explored in further detail hereunder is one of the mechanisms utilised to improve shelf stability and gastric stability. Shelf stability is of relevance particularly in the least developed countries where maintenance of the cold chain for the transport and storage of drugs is difficult if not impossible and where the stabilisation of the environment to prevent degradation by oxygen and moisture is often times not possible.

The nutraceuticals market is also a highly relevant economic market with an estimated value of USD 454 billion in 2021. The projected probiotics market globally in 2023 is estimated at USD 57.2 billion with a market growth rate of 7-8%. The reason for the size of this market is that probiotics and phytochemicals, to name a few, are well recognised for the health benefits that they impart on both humans and animals. This is particularly relevant to humans given aging populations showing a higher mean age than in previous decades. In animals probiotics-based feed additives, play a critical role in maintaining and improving the gut health in livestock and preventing and controlling enteric pathogens, the microbial ecology of said animal gut having an integral role in the productivity of the animal. Nutraceuticals are also known to have multiple therapeutic properties including anticancer, antithyroid, antioxidant, anti-inflammatory, cardio-protective, antimicrobial, and many other benefits. What distinguishes the most therapeutically effective nutraceuticals from others available on the market is that they have enhanced delivery systems. Phytochemicals are also widely used in the nutraceutical market. However, many phytochemicals, are poorly absorbed due to low bioaccessibility when digested and correspondingly low bioactivity and efficacy. This low efficacy has an impact on the commercial potential of such chemicals and hence it would be advantageous to identify manners in which the efficacy of the same can be improved.

Nutraceuticals contain natural compounds or derivatives thereof as do many pharmaceutical drugs. Unfortunately, natural compounds generally have low bioavailability, limited solubility, are quickly metabolised, are gastrically sensitive and have low shelf stability. They typically degrade when exposed to harmful environments during processing, storage and/or consumption, for example oxygen, moisture, and high temperatures. Degradation could mean complete degradation of the drug such that the same has no efficacy or partial degradation which would result in highly reduced drug viability neither of which is desirable given both the issues of access to drugs and the expense associated with obtaining and/replacing them. A longer shelf stability would therefore assist with both access to medicines and the costs associated with purchasing the same as medicines could be stored for longer periods and remain stable which would reduce concomitant costs associated with having to dispose of expired medicines and purchase new ones.

Natural compounds are also highly sensitive to gastric degradation once digested which is not beneficial if the same are intended for absorption in the intestines which directly influences the bioavailability of the same. With reduced bioavailability higher concentrations of ACs will be required to make a drug effective which would mean larger costs associated with producing the same which costs will invariably be passed on to the patient. Higher costs associated with medicines make the same simply unaffordable in medium to lower income countries and hence means that access to life saving treatments and nutritional supplements to address malnutrition is simply not possible.

To address the abovementioned problems encapsulation of labile ACs is used. Encapsulation is the process of stabilization of ACs through the structuring of systems capable of preserving their chemical, physical, and biological properties, as well as improving their release profiles under desired conditions. Typically, ACs are encapsulated before being introduced into the final dosage form of the medicament which medicament is administered to a human and/or animal in need thereof. As alluded to above, such encapsulation is aimed at shielding the compound from detrimental interaction with the ambient environment and/or gastric acid after ingestion as well as to mask undesirable tastes; to improve solubility and/or bioavailability and to assist in controlling the release profile of the same.

Bioavailability, release profile and solubility are also of importance as these factors ultimately influence the concentration of AC required to achieve therapeutic efficacy. Adequate encapsulation and reduced gastric degradation will invariably improve the pharmacokinetics and bioavailability of the AC which improves the therapeutic efficacy of the same meaning lower concentrations of AC will be required to achieve therapeutic efficacy and correspondingly lower costs.

Drug solubility can be also a limiting factor with more than 70% of newly discovered drugs being water insoluble. Decreased aqueous solubility results in decreased bioavailability and biodistribution of the AC and meaning a higher concentration of AC would be required to achieve the desired therapeutic effect implying larger costs with respect to manufacturing.

Another possible consequence of lower AC concentrations in drugs is decreased cytotoxicity (from said lower dose) which is of relevance in respect of pharmaceutical products with potentially cytotoxic APIs. For example, chemotherapeutic drugs are currently delivered systemically which results in side effects that are associated with exposing non-cancerous cells and/or tissues to such drugs. Systemic exposure to an API can lead to a reduced ability to treat various diseases given the side effects or drug toxicity associated with such systemic exposure. Lower concentrations of API and increased bioavailability would therefore be advantageous as lower doses would result in decreased cytotoxicity and concomitant side effects.

Encapsulation is utilised as an alternative to the use of synthetic preservatives and flavourants. This is advantageous given that in recent times there has been a focus on the possible detrimental health effects and environmental effects associated with the use of such compounds with the world generally moving towards the adoption of “green processes” and renewable energy resources.

During the process of encapsulation, the AC or a combination of ACs forming the so-called “core” is coated so that a protective “shell” is formed around the same. The shell or encapsulation/carrier material can be in the form of solid, liquid droplets, and/or gas bubbles to encapsulate a liquid or gas inside as a core. The chemical composition of the core and encapsulation material would invariably differ with respect to application for which the end-product/drug is used. In the food industry, for example, essential oils may be included in the core with shell materials of whey protein, gum arabic, maltodextrin, etc. In the pharmaceutical industry where APIs are to be protected the core may comprise influenza virus, stem cells, DNA, and insulin and shell materials may include polymethylmethacrylate.

With respect to shell material utilised in the pharmaceutical and/or nutraceutical industry lipids provide some advantages. Various examples of lipids being used in encapsulation have been shown and in many of these examples only lipids have been utilised rather than a combination of compounds. Lipids can protect probiotics from gastric acid (as they are stable in acidic environments) and allow for the release of the same in the intestines via emulsification or lipolysis. Furthermore, lipids are food-safe, GRAS (generally recognised as safe) certified, are low-cost compounds and are suitable for use in medicines intended for the treatment of humans and/or animals. However, there are several disadvantages associated with the use of lipid shells, including: that they are highly crystalline which may lead to expulsion of encapsulated ACs outwards towards the surface of the microparticles; they are non-polar which makes them poorly compatible or incompatible for use in encapsulating polar ACs and they are prone to micro-cracking which results in poor shelf stability. It is possible to ameliorate some of these challenges by introducing additives to disrupt the crystallinity of the lipids and to improve their compatibility with polar ACs. However, none of these strategies assist with increased shelf stability.

Clay additives (probiotic binding substrates) and/or the inclusion of a surfactant can assist in enhancing lipid stability during encapsulation which allows for better shelf stability. Furthermore, an additive with desiccant properties could also be added to improve shelf stability. However, this is not ideal as none of these additives contribute to gastric stability (gastric resistance) given their hydrophilic nature. For example, desiccants are highly soluble and able to dissolve in the gastric environment leading to the release of the AC before the same reaches the target site for release.

In the current probiotic market “ProbioFerm” is of relevance. This company has developed a product which has been coined Durabac™. Durabac™ is an encapsulation technology that seeks to achieve enhanced shelf and gastric stability using methodology and formulations which differ from those set out herein in accordance with the subject invention as the same utilise a coating process for encapsulation. Albeit that this currently commercially available technology has provided for some improvement as regards the challenges associated with encapsulation there is a definite need for further improvement and optimisation.

In the pharmaceutical and/or nutraceutical industries such encapsulation may also affect drug delivery and thereby speaks to drug delivery systems as well as to mechanisms of preservation. Briefly, drug and nutraceutical delivery is a broad biomedical field that encompasses approaches, formulations, manufacturing techniques, storage systems, and technologies involved in transporting an AC to its target site and/or releasing the same at said target site to achieve the desired therapeutic effect. Delivery vehicles are utilised in drug delivery systems which concern the delivery of conventional APIs but also to deliver nutraceuticals. Site specific and naturally based drug delivery which can be aided by encapsulation influences bioavailability and therefore potential drug side effects, as set out above.

ACs that are encapsulated may be released from the core via controlled, alternatively uncontrolled release which influences the release profile of the AC. Controlled release is desirable and is affected by crack propagation of the polymeric shell when the same is exposed to mechanical stimulation thereby allowing the liquid core to be released. Such controlled release at the desired therapeutic site is highly desirable as regards therapeutic efficacy and required AC concentrations to achieve the same.

In addition to use in the pharmaceutical and nutraceutical industries encapsulation is also used in the food industry to add flavour profiles and reduce processing steps and in the agricultural industry for encapsulating biopesticides, fertilizers, and other agrochemicals that allow growers to precisely control the conditions under which the AC is released. This controlled release is of relevance with respect to biopesticides as such controlled release allows for a much more limited exposure of the environment to the biopesticide thereby providing “greener” solutions as regards the application of biopesticides. This technology has also found application in the cosmeceuticals industry for the protection of ACs. Accordingly, it will be appreciated by a person skilled in the art that the application of the invention is by no means limited to the pharmaceutical and/or nutraceutical market but that the same has broad applicability.

Currently known methods of encapsulation can be divided in three possible modalities:
Chemical encapsulation: This is achieved by coacervation, molecular inclusion and cocrystallization.
Physical/mechanical encapsulation: this is achieved by spray-drying, extrusion, freeze-drying and vacuum drying, spray-cooling, or chilling, and fluidized bed coating.
High- and low-energy emulsification techniques: Two types of emulsion are formed as part of the encapsulation process. The fundamental difference between these two types is the droplet size of said emulsions being formulated and treated to provide for encapsulation.

In addition to gastric sensitivity natural compounds are also sensitive to the process utilised for encapsulation which may result in decreased shelf stability and/or lower therapeutic efficacy as the conventional processes utilised for encapsulation expose the labile ACs to harsh conditions (extreme temperatures, solvents, water and oxygen) that are not conducive to the maintenance and protection of such labile ACs.

An example of an encapsulation system reliant on the use of high temperatures and/or exposure to solvents during the process of encapsulation is given in WO2014166994 A1 which describes nano-micro delivery systems for the oral delivery of. ACs which system envisages the use of predominantly lipids but also the inclusion of polymers. However, the invention set out in the abovementioned patent application does not include the use of additional additives to stabilise the encapsulation material shell which implies that shelf stability will be impacted given that there is no stabilising compound to assist in providing protection against ambient environmental factors.

In U.S. Pat. No. 8,637,104B2 a microencapsulate and the process for the manufacture thereof is described. The method utilised for the formulation of the microencapsulate, unlike the method utilised in WO2014166994 A1, is reliant on the use of a supercritical fluid and the antisolvent (SAS) effect associated therewith. However, this microencapsulate is a solely lipid-based encapsulate and hence albeit that the lipid composition provides for gastric stability there is no provision made within this system for shelf stability. It is important to note that SAS is associated with the use of a supercritical fluid as an antisolvent which differs fundamentally from Particles from Gas Saturated Solutions (PGSS) which is described hereunder in that PGSS utilises the supercritical fluid as a solvent or plasticiser.

With respect to the currently known AC encapsulation systems and methods of formulating the same it would be highly advantageous to provide for an encapsulation system and method for formulating the same that at least in part addresses the problems associated with the current systems and methods used in the art.

### Definitions

For the purposes of interpretation of this specification the following definitions shall apply:

Abbreviations: the abbreviations for various materials and/or experimental techniques or methodology utilised in the examples in respect of the subject invention is given in the summary table below;

| Material/Technique | Abbreviation |
|---|---|
| Carbon dioxide | CO₂ |
| Myristic Acid | MA |
| Oleic Acid | OA |
| Lecithin | Lec |
| Palm Stearin | PS |
| Probiotic Multi-strain | GO-01 |
| Bifidobacterium bifidum | Bb-2 |
| Lactobacillus reuteri | Lr-2 |
| Phosphate Buffered Saline | PBS |
| Polyvinylpyrrolidone | PVP |
| Rosemary Powder Extract | Inolens 4 |
| Scanning Electron Microscopy | SEM |
| Simulated Gastric Fluid | SGF |
| Simulated Intestinal Fluid | SIF |
| Supercritical carbon dioxide | sc-CO₂ |

Active Compound(s) (AC): is the active compound(s) contained in a drug, pesticide and/or a nutraceutical that provides the desired therapeutic outcome;

Active Pharmaceutical Ingredient (API): is the active ingredient that makes a drug product effective and provides the pharmacological activity desired. This is of particular relevance in relation to the pharmaceutical industry;

Amphiphile: is a compound that has both hydrophilic and hydrophobic parts;

Cosmeceutical: is a cosmetic that has or is claimed to have medicinal properties;

Colony forming units (CFU, cfu or Cfu): is a unit which estimates the number of microbial cells (bacteria, fungi, viruses) in a sample that are viable and may also be denoted in colony forming units per gram (cfu/g) where the number of cfu is measured in relation to the unit mass of a substance of interest;

Dosage Form: drugs in the form in which they are marketed for use, with a specific mixture of active compounds and inactive components (excipients), in a particular configuration (such as capsules, tablets etc) and a particular dose;

Drug: a medicine or other substance which has a physiological effect when ingested or otherwise introduced into the body. This is considered to be inclusive of both pharmaceutical and nutraceutical products;

Encapsulation: is the process of stabilization of active compounds through the structuring of systems capable of preserving their chemical, physical, and biological properties, as well as improving their release or delivery under established or desired conditions;

Eutectic system/Eutectic mixture: is a homogeneous mixture of two or more substances that form a super-lattice crystal structure that melts or solidifies at a single temperature (seen at the eutectic point on a phase diagram) that is lower than the melting point of any of the constituents. Eutectics are formed via a liquid-solid transitions and may be formed between metals, glasses, polymers, lipids and drugs to name but a few examples;

Eutectoid: is a system bearing the same properties as a eutectic system save that a eutectoid is formed via a solid-solid transition and not via a liquid solid transition;

Gastric stability shall be used interchangeably with Gastric resistance said terms referring to the ability of a material to remain stable alternatively avoid gastric attack and degradation in a gastric or simulated gastric environment;

GO-01 Strain (GO-01): is a Multi-strain probiotic stock comprising a combination of the following strains: Lactobacillus acidophilus, Lactobacillus plantarum, Lactobacillus reuteri, Lactobacillus casei, Lactobacillus rhamnosus, Lactobacillus fermentum, Lactobacillus salivarus, Lactobacillus paracasei, Lactobacillus bulgaris, Lactobacillus helviticus, Bifidobacterium lactis, Bifidobacterium infantis, Bifidobacterium breve, Bifidobacterium bifidum, Bifidobacterium longum and Streptococcus thermophilus (Supplied by Biogrowing Company Ltd).

Hypo/Hyper Eutectic Mixture: is a mixture having a composition that If the places it to the left of the eutectic point on a phase diagram, then it is (hypoeutectic) or to the right of the eutectic point (hypereutectic).

Medicine/Medicament: is a substance used for medical treatment of humans and/or animals and shall be used interchangeably with drug;

Nutraceutical: means nutritional substances isolated from natural foods which are used not just for the nutritional purpose but also used for therapeutic or physiological benefits. They include a wide range of compounds from bioactive peptides, phenolic compounds, flavonoids, carotenoids, lipids, minerals, vitamins, amino acids, antioxidants, plant metabolites, fortified foods and combinations of these ingredients.

Phytochemical: means bioactive nutrient plant chemicals in fruits, vegetables, grains, and other plant foods that may provide desirable health benefits beyond basic nutrition for example, to reduce the risk of major chronic diseases;

Supercritical (sc) fluid: is any substance at a temperature and pressure above its critical point, where distinct liquid and gas phases do not exist, but below the pressure required to compress it into a solid.

### Object of the Invention

It is an object of this invention to provide for an encapsulation system and method for formulating the same, for use in encapsulating one or more ACs to, at least in part, ameliorate the challenges associated with currently utilised methods and systems that do not provide for both gastric and shelf stability.

### SUMMARY OF THE INVENTION

In accordance with a first aspect of the invention there is provided an encapsulation system, for encapsulating at least one active compound (AC), including an encapsulating material selected from the group comprising: a eutectic mixture, a eutectoid, a hyper eutectic mixture and a hypo-eutectic mixture and at least one additive, the encapsulating material and at least one additive being homogenously combined with the AC in a solvent medium comprising a supercritical compound to form a matrix which encapsulates the AC so that, in use, the AC is protected from both ambient environmental factors and gastric attack, thereby providing for shelf stability and gastric stability respectively.

There is also provided that the AC is selected from the group comprising probiotics, bacteria, yeast, vitamins, minerals, phytochemicals, phytogenic compounds, essential oils, flavourants, biological actives and APIs.

There is further provided that the biological actives may include, but not be limited to peptides, proteins, enzymes and antibodies.

There is also provided that the encapsulating material is formulated using a combination of at least two lipids, alternatively the combination of at least one lipid and one polymer wherein the at least one lipid remains intact upon exposure to gastric conditions in the stomach of a human and/or animal thereby ensuring that the matrix is resistant to gastric attack so that the same is only solubilised by emulsification and/or lipolysis which occurs in the intestine of the human and/or animal.

There is further provided that the encapsulating material upon combination with the at least one additive provides for stability of the at least one additive within the matrix.

There is also provided that the additive may be selected from the group comprising: hygroscopic compounds and polymeric amphiphiles or a combination of the aforementioned.

There is further provided that the hygroscopic additive may be a desiccant and may be selected from the group comprising: natural and synthetic polymers, sugars, clay, salt and silica gel; the hygroscopic additive functioning, in use, to lower the water activity around the AC thereby protecting the same from environmental humidity and hence providing shelf stability with respect to a medicament incorporating said encapsulated AC.

There is further provided that the polymeric amphiphilic additive is selected from the group comprising: peptides, phospholipids, surfactants, fatty acids and glycolipids; the polymeric amphiphilic additive disrupting the crystalline structure of lipids contained in the matrix and/or influencing the molecular polarity of the matrix and/or assisting in the prevention of the formation of micro-cracks in the lipid all of which would be detrimental to both the shelf stability and carrier properties of the matrix.

There is further provided that the increased molecular polarity of the matrix having been induced through the action of the polymeric amphiphile allows for the encapsulation of polar active compounds by the encapsulation system.

There is also provided that the supercritical compound is selected from the group comprising: supercritical (sc-) carbon dioxide, water, hexane, methane and ethanol but in a preferred example embodiment of the invention the sc-compound is sc-CO₂.

There is also provided that the supercritical compound is produced utilising pressures and temperatures at or higher than the defined critical temperatures and pressure for said compound.

There is further provided that the supercritical compound is produced in anoxic and anhydrous conditions.

There is also provided that the encapsulating material when combined with the active compound forms microparticles having a size of about 5 to about 150 μm.

In accordance with a second aspect of the invention there is provided a method of formulating the encapsulation system in accordance with the first aspect of the invention, the method including producing supercritical fluid through the application of at least critical temperature and pressure to said fluid to produce a supercritical solvent, mixing the supercritical solvent with a combination of encapsulating material, at least one additive and active compound (AC) or compounds as provided for in accordance with the first aspect of the invention so as to allow the mixture to rapidly cool upon the evaporation of the supercritical solvent, said rapid cooling providing a temperature lower than the solidification temperature of the combination of encapsulating material, at least one additive and AC which results in the atomisation and solidification of the combination of the encapsulating material, at least one additive and AC resulting in the encapsulation of the AC or ACs.

In accordance with a third aspect of the invention there is also provided that the combination of the encapsulating material and AC utilising the process in accordance with a second aspect of the invention results in the formation of a microencapsulated AC.

In accordance with a fourth aspect of the invention there is provided a medicament produced in accordance with the methodology of the second aspect of the invention for use in delivery of said AC to a human or animal in need thereof.

There is also provided that the medicament may be indicated in the treatment of diseases and contain an API and/or APIs, alternatively that the medicament may be indicated for use as a nutraceutical, cosmeceutical, feed additive and/or fortified food to provide health benefits to said human and/or animal and may contain compounds appropriate for these purposes.

There is further provided that the nutraceutical shall provide a dosage of at least, 10⁷ cfu/g.

In accordance with a fifth aspect of the invention there is provided a method of treatment of the human and/or animal body by administering the medicament as provided for in accordance with the fourth aspect of the invention to a human and/or animal in need thereof.

In accordance with a sixth aspect of this invention there is provided an encapsulation system in accordance with the first aspect of the invention for use in the agricultural industry for the encapsulation of but not limited to biopesticides to provide for better specificity as regards the application of the same.

### BRIEF DESCRIPTION

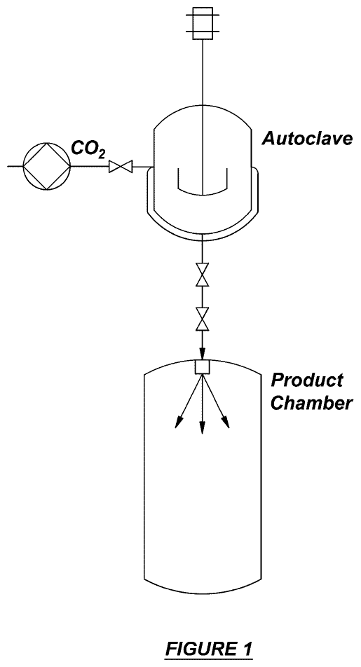

FIG. 1 is the apparatus utilised in the method of formulating the encapsulation system of the invention;

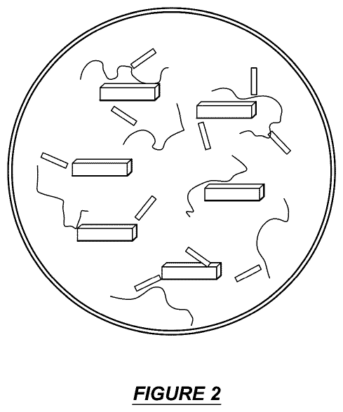

FIG. 2 is a diagrammatic representation of the encapsulation system of the invention;

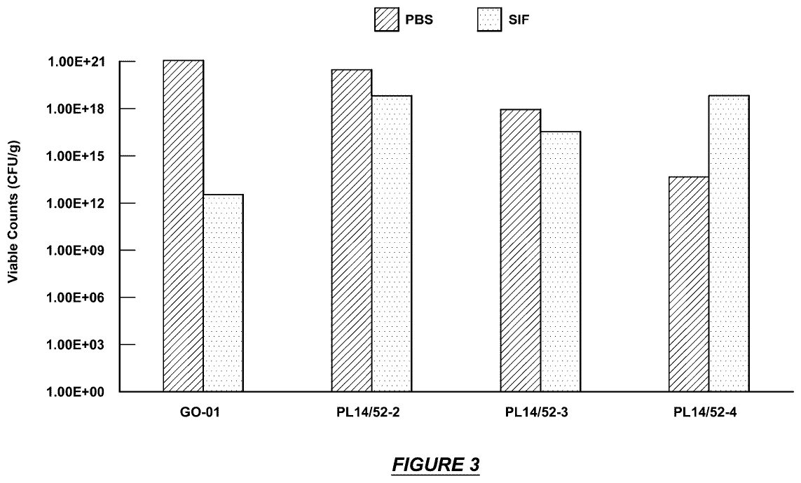

FIG. 3 is a graph showing the viable counts in cfu/g for encapsulated GO-01 and unencapsulated GO-01 in PBS and SIF;

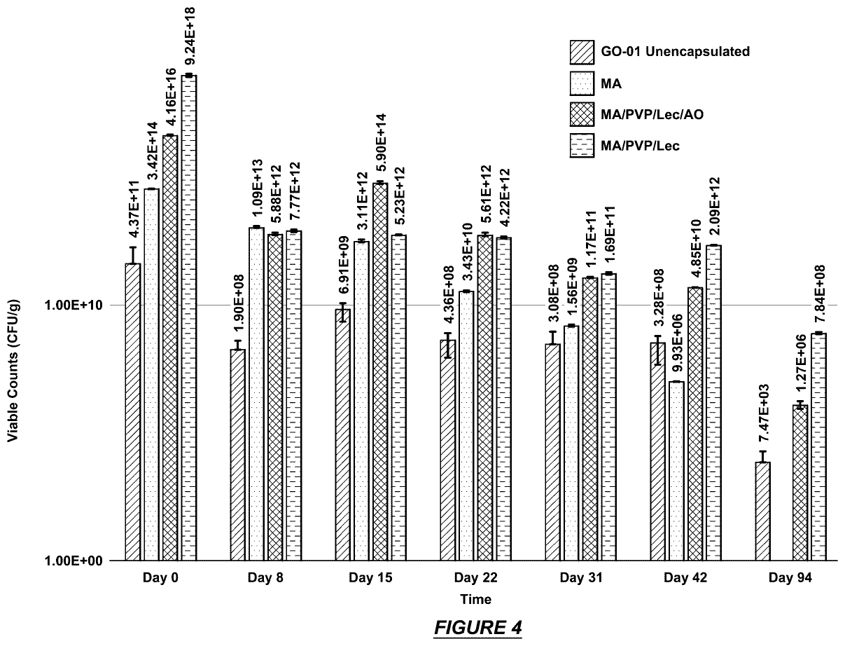

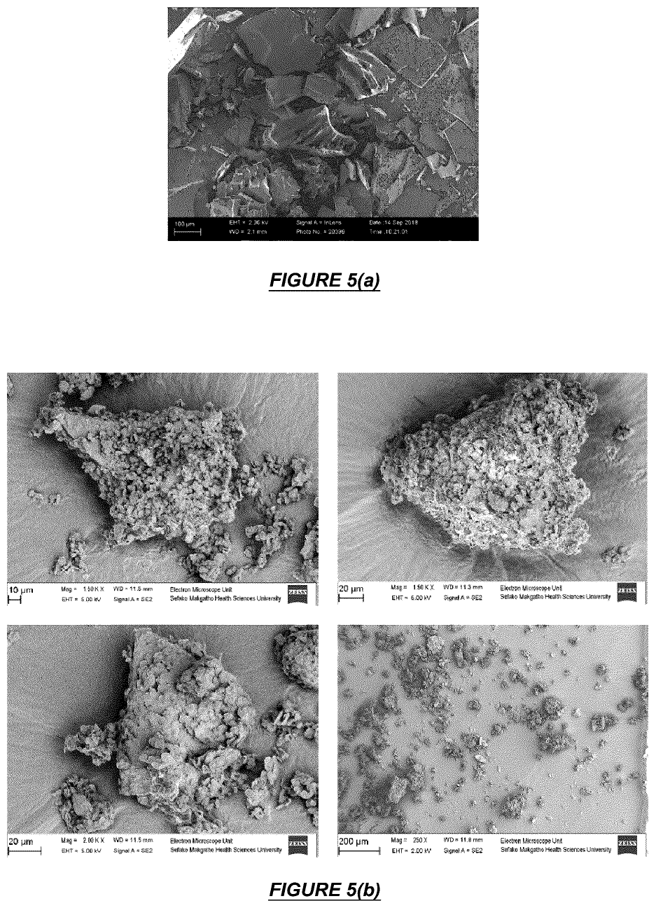

FIG. 4 is a graph showing the viable counts in cfu/g for formulations of encapsulated GO-01 and unencapsulated GO-01 over a period of 94 days which is indicative of the shelf stability for the same;

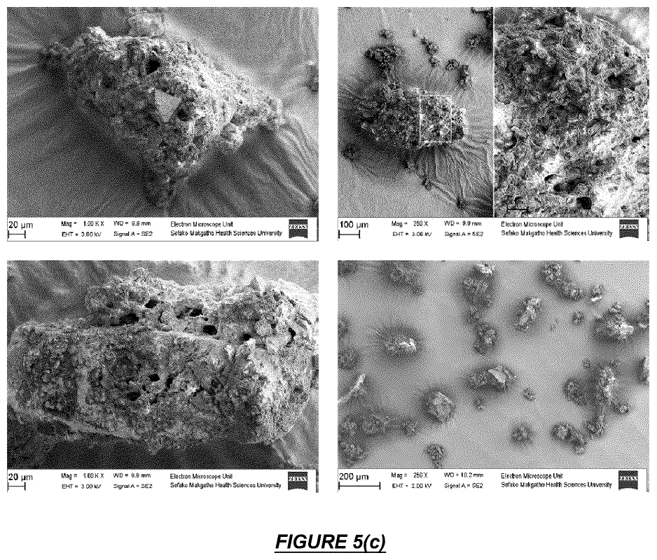

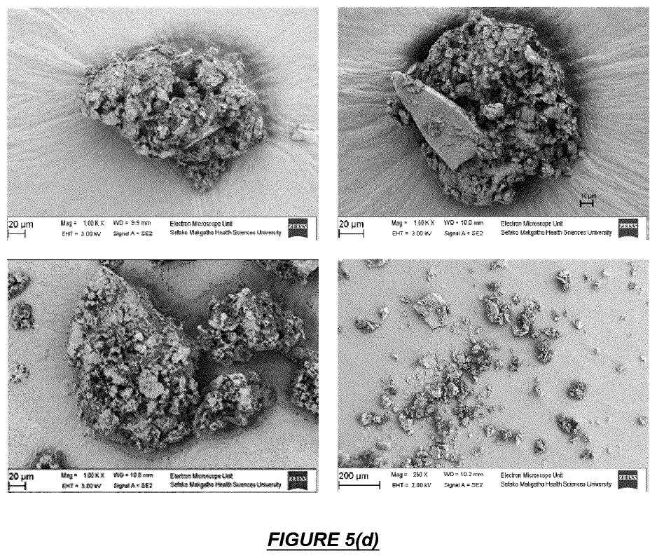

FIG. 5(a) are cryo-SEM images of the unencapsulated GO-01;

FIG. 5(b) are cryo-SEM images of encapsulated formulation PL14/52-2;

FIG. 5(c) are cryo-SEM images of encapsulated formulation PL14/52-3;

FIG. 5(d) are cryo-SEM images of encapsulated formulation PL14/52-4;

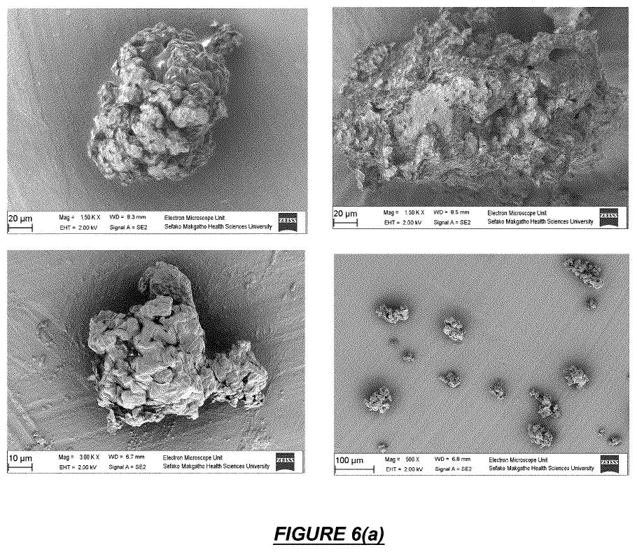

FIG. 6(a) are cryo-SEM images of encapsulated formulation PL14/57-3;

FIG. 6(b) are cryo-SEM images of encapsulated formulation PL14/57-4;

FIG. 6(c) are cryo-SEM images of encapsulated formulation PL14/57-5;

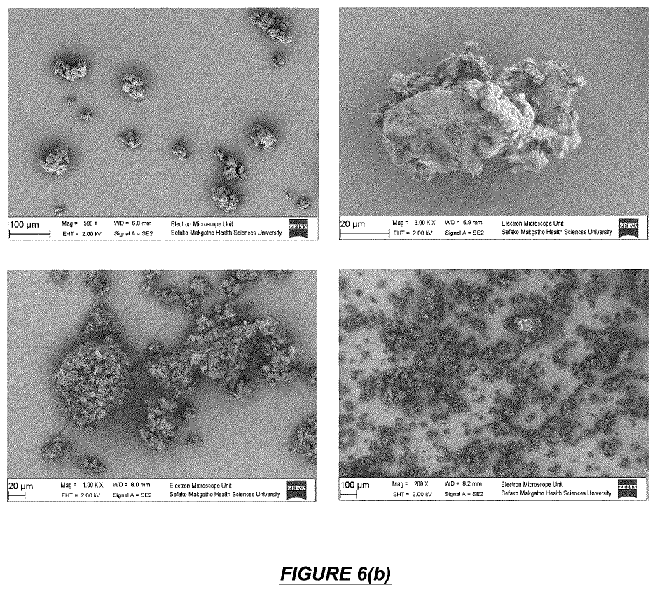

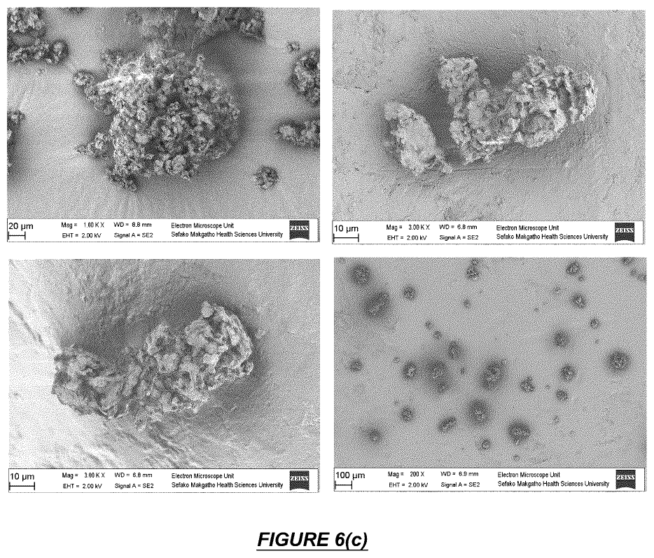

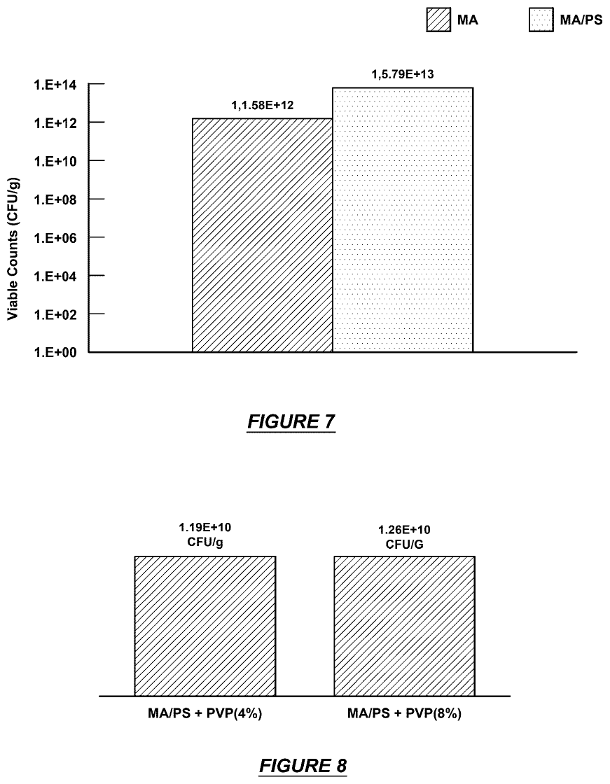

FIG. 7 is a graph showing the viable counts in cfu/g for various lipid-lipid encapsulated hybrids in SIF;

FIG. 8 is a graph showing the viable counts in cfu/g for various lipid-lipid-PVP formulations after exposure to SGF;

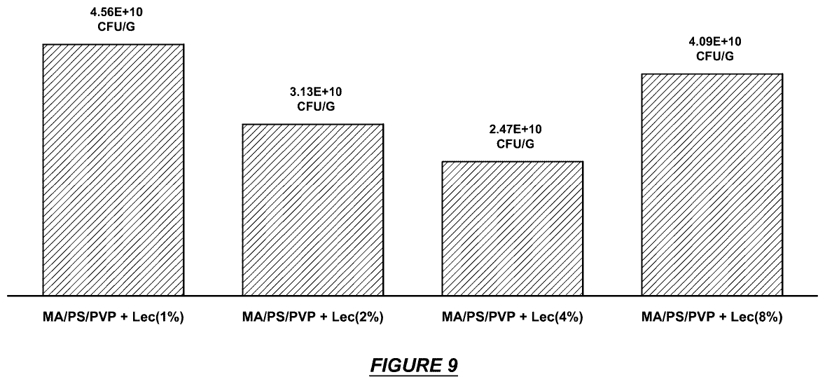

FIG. 9 is a graph showing the viable counts in cfu/g for various lipid-lipid-PVP-Lec formulations after exposure to SGF;

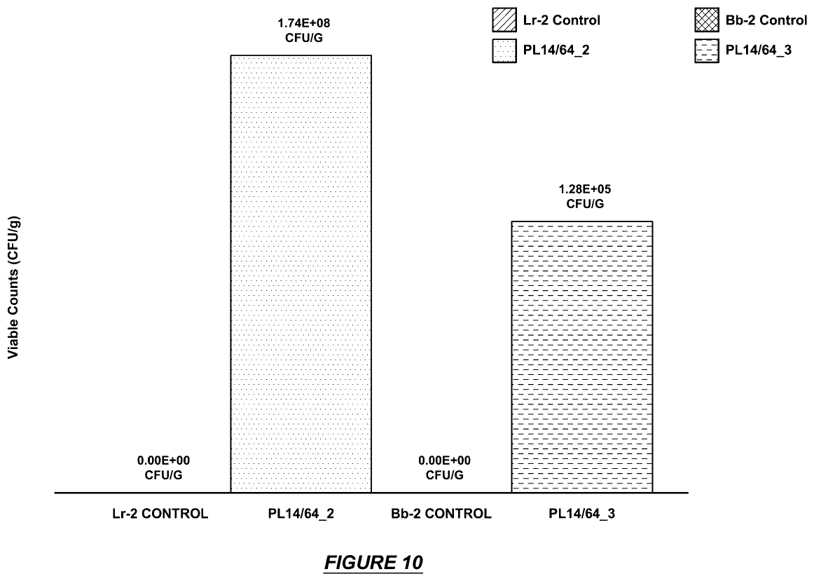

FIG. 10 is a graph showing the results of viable counts in cfu/g in respect of gastric resistance tests for unencapsulated Bb-2 and Lr-2 in comparison to Bb-2 and Lr-2 encapsulated in lipid-lipid-PVP-Lec formulations;

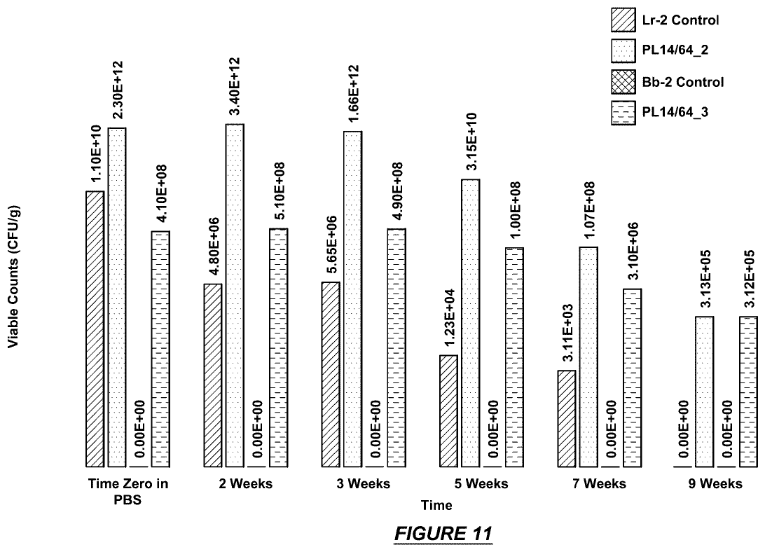

FIG. 11 is a graph showing the results of viable counts in cfu/g in respect of shelf stability tests for unencapsulated Bb-2 and Lr-2 in comparison to Bb-2 and Lr-2 encapsulated in lipid-lipid-PVP-Lec formulations;

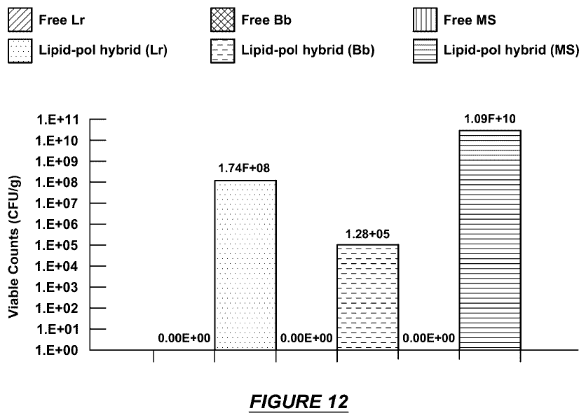

FIG. 12 is a graph showing the gastric exposure survival rate of probiotics encapsulated using the encapsulation system of the invention as compared to the survival rate of free probiotics wherein viability is measured in cfu/g;

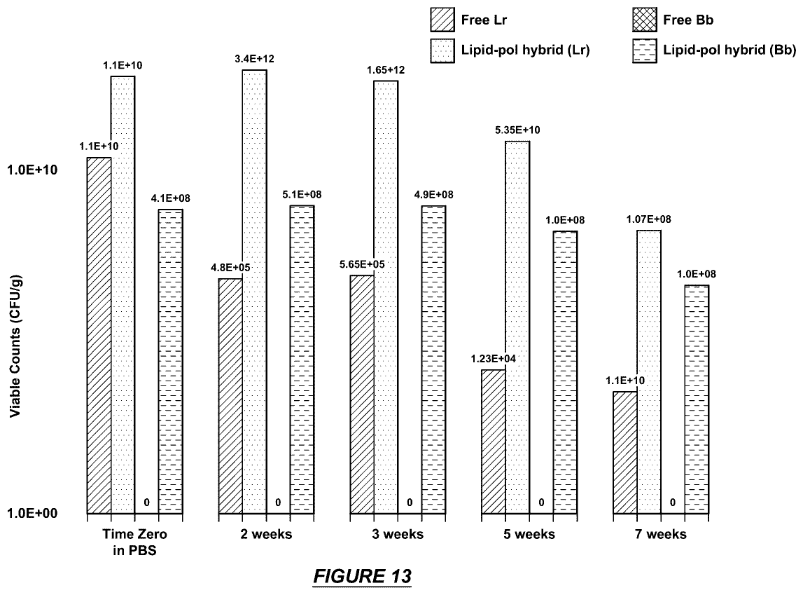

FIG. 13 is a graph showing the shelf stability of probiotics encapsulated using the encapsulation system of the invention as compared to the shelf stability of free probiotics wherein viability is measured in cfu/g; and

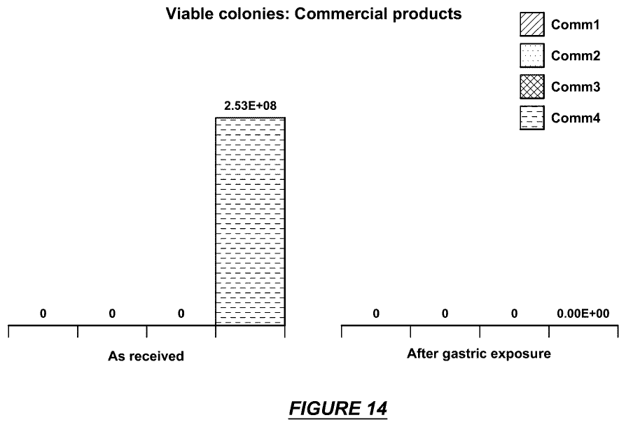

FIG. 14 is a graph showing the shelf stability (shown prior to gastric exposure) and the simulated survival rate (shown post gastric exposure) of commercially available products Comm 1 to comm 4 prior to and post exposure to simulated gastric conditions wherein viability is measured in cfu/g.

### EXAMPLES

The invention shall now be described with reference to the examples given hereunder. However, it will be appreciated by a person skilled in the art that alternative formulations that still fall within the scope of the invention, as claimed, are possible and, as such, the examples given herein by no means limit the scope of the invention.

### Example 1: Process for the Encapsulation of AC in Accordance with the Invention

### Materials

Apparatus for performing the method to produce the encapsulation system as shown in FIG. 1;
a labile or sensitive ACs selected from the group comprising: probiotic, bacteria, yeast, vitamins, minerals, phytochemicals, phytogenics, essential oils, flavourants, biological active and/or APIs;
a lipid selected from the group comprising fats, waxes, fatty acids, fatty acid esters and/or fatty alcohols. In a preferred example embodiment of a formulation in accordance with the invention OA and/or Lec and/or MA are used;
a eutectic, hypoeutectic or hypereutectic mixture of two or more materials selected from the group comprising lipids and/or polymers;
an additive comprising hygroscopic compound and/or desiccant selected from the group comprising: natural and synthetic polymers, sugars, clay, salt and silica gel;
an additive comprising polymeric amphiphile selected from the group comprising: peptides, phospholipids, surfactants, fatty acids and/or glycolipids;
a medium comprising a compound in a supercritical state produced under oxygen and moisture free (anoxic and anhydrous) conditions through the application of the specified conditions of pressure and temperature which are at or higher than the defined critical temperature and pressure for said compound. The sc-compound is selected from the group comprising: CO₂, water, hexane, methane and ethanol and once treated forms supercritical (sc-) CO₂, water, hexane, methane and ethanol;

### Method

The encapsulation process of the invention utilises the methodology of Particles from Gas Saturated Solutions (PGSS) where the medium within which the encapsulation occurs is in the supercritical state.

Reaction parameters: the reaction is carried out in a moisture free environment at a pressure of approximately 10 000 KPa and 40° C. with the mixture being subject to continuous stirring (with baffles in place).

By way of example, supercritical carbon dioxide (sc-CO₂) is referenced. The sc-CO₂ is produced in an anoxic, anhydrous environment with stable conditions of pressure and temperature. The PGSS involves solubilising the compressible medium (in this instance the CO₂ which becomes a supercritical fluid) in the substance to be micronized. The encapsulating material in the subject invention comprises a homogenised mixture of lipids, a hyper eutectic or a hypoeutectic mixture; the hygroscopic compound or desiccant and the polymeric amphiphile that combine to form the shell of the microcapsule which shell encapsulates the active compound or compounds. In a preferred example embodiment, the encapsulating material forms a matrix with the AC rather than a shell and core configuration.

The sc-CO₂ is the able to liquify the encapsulating material in this supercritical soluble state. ‘When the sc-CO₂ encapsulating material and API is fed into the atomisation chamber the gas (CO₂) rapidly expands and evaporates. This, results in the rapid cooling of the solution due to the Joule-Thompson effect and/or the evaporation and volume expansion of the gas. This rapid cooling drops the temperature of the solution to below the solidification temperature of the solute (AC and encapsulating material mixture) contained therein resulting in the atomisation and solidification of the encapsulating material/AC mixture and the formation of a microencapsulated AC in accordance with the third aspect of the invention as shown in FIG. 2.

It has been shown that sc-CO₂ as when utilised in this method with eutectic, alternatively hypoeutectic, further alternatively hypereutectic mixtures provide much lower melting temperatures for lipids than are ordinarily seen with conventional processes meaning that much lower processing temperatures are required to achieve encapsulation. Lower processing temperatures are highly favourable as active compounds are oft times very sensitive to high temperatures which ultimately lead to the decreased efficacy of the same simply due to the methodology utilised for their encapsulation.

No toxic or flammable organic solvents are utilised during the application of this process thereby distinguishing the process used to produce the encapsulation system of the invention from encapsulation processes currently used in the art. This process is highly beneficial in that given that no potentially harmful solvents are utilised and given that the process is conducted at lower temperatures, both of which make the process significantly more environmentally friendly than currently utilised processes in the art.

### Results

The performance of the method results in the formulation of a microencapsulated AC or ACs. The ACs are contained in the core of the microcapsule and surrounded by the encapsulating material, alternatively the compounds are encapsulated such that the ACs and encapsulating material forms a matrix.

The lipid or lipids contained in the encapsulating mixture and in the lipid-lipid eutectic mixture have good barrier properties to moisture and oxygen both which are required for stability enhancement of labile actives. In addition, many lipids remain intact upon exposure to acidic environments (such as gastric juices) while they are disintegrated in intestinal juices via emulsification and/or lipolysis. Thus, they could potentially protect labile actives when passing through the stomach and allow for release in the intestines where they impart their health benefits. The eutectic, hypoeutectic or hypereutectic mixture functions to stabilise the highly water-soluble additives in the lipid matrix providing for better shelf stability and in combination with the lipid or lipids contained in the encapsulating mixture for better gastric stability. Furthermore, the introduction of a polymeric amphiphilic additive in the encapsulating mixture functions to provide enhanced polar compound compatibility in the instance that the ACs being encapsulated are polar hydrophilic compounds.

### Example 2.1: Formulations Comprising Lipid-Polymer Hybrids

The formulations analysed are shown in Table 1 hereunder:

TABLE 1
Formulations

| Reference | Ingredients | Loading (wt %) |
|---|---|---|
| PL14/52-2 | Myristic Acid (ma) | 87 |
| | MS Probiotic | 10 |
| | Antioxidant mix* (AO) | 3 |
| PL14/52-3 | Myristic Acid | 69.6 |
| | Polyvinylpyrrolidone | 8.7 |
| | (PVP) | |
| | Lecithin (Lec) | 8.7 |
| | MS Probiotic | 10 |
| | Antioxidant mix* | 3 |
| PL14/52-4 | Myristic Acid | 71.7 |
| | Polyvinylpyrrolidone | 9 |
| | (PVP) | |
| | Lecithin | 9 |
| | Ms Probiotic | 10.3 |

*Antioxidant mix: α-tocopherol; L-ascorbic acid; Inolens 4 (1:1:1 ratio)

### Method

PGSS was carried out to achieve the encapsulation of the formulations under the following processing conditions:
Temperature: 40° C.
Pressure: 10 000 KPa
Process Time: 1 hour
Stirring: continuous at “300” with baffles

The micronisation of the formulations was assessed prior to the conducting of the additional assessments referred to hereunder and it was found that full micronisation for all the formulations was achieved. This was indicated by the fact that no residual product remained in the mixing vessel after the reaction.

### Example 2.2: Assessment of Stability in SIF

The abovementioned encapsulated formulations together with an unencapsulated control (GO-01) were assessed for viability in PBS and SIF by conducting viability counts for the same after incubation for 48 hours under aerobic conditions.

### Results

The results of the viability counts conducted is shown in FIG. 3, wherein GO-01 represents the unencapsulated control formulation which showed a high viability in PBS but a much lower viability when tested in SIF.

Encapsulated samples PL14/52-2 and PL14/52-3 showed approximately equal viability in PBS, SIF, and PL14/52-4 showed much better viability in SIF than in PBS.

It was concluded that PL14/52-4 allowed for significantly better release of probiotics in an SIF environment than the other encapsulated samples tested.

### Example 2.3: Assessment of SGF Survival

The abovementioned encapsulated formulations together with an unencapsulated_control (GO-01) were assessed for viability in SGF by conducting viability counts for the same after 2 hours of shaking in SGF (pH1.2) and 4 hours of shaking in SIF (pH6.8).

### Results

The results of this assessment showed that PL14/52-2 which contained only MA showed good viability in SGF which is indicative of potentially good gastric protection whilst the other formulations did not.

### Example 2.4. Assessment of Shelf Stability

Encapsulated formulations with GO-01 and encapsulating material MA; MA/PVP/Lec/AO and MA/PVP/Lec together with an unencapsulated control (GO-01) were assessed for shelf stability over a period of 94 days by conducting viability counts for the same. The formulations having been stored at 25° C. and 50% relative humidity in laminated foil sheets.

### Results

The results of the assessment of shelf stability are shown in FIG. 4 wherein it can be seen that albeit that there is a decrease in shelf stability for all formulations over a period of 94 days formulations containing a combination of MA/PVP/Lec showed the greatest shelf stability whilst formulations containing only MA showed much lower shelf stability and degraded completely by 94 days.

It was assumed that the MA when utilised alone allowed for a certain amount of atmospheric moisture to reach the probiotic resulting in decreased viability over time.

### Example 2.5: SEM Analysis

The lipid/probiotic microparticles were visualised using Cryo-SEM to provide an image of the probiotic flakes with different lipid formulations. The SEM images resulting from this analysis are shown in FIGS. 5(a) to 5(d).

As can be seen in the SEM images in general a good lipid coating on to the GO-01 was seen in all encapsulated formulations albeit that not all flakes were covered. The morphology of the encapsulated formulations being more fluid rather than particulate especially for PL14/52-3 which it was assumed is to be associated with the surfactant properties of the included lecithin.

### Example 3.1: Formulations Comprising Lipid-Lipid Hybrids

The formulations analysed are shown in Table 2 hereunder:

TABLE 2
Formulations

| Reference | Ingredients | Loading (wt %) |
|---|---|---|
| PL14/57-3 | MA | 45.15 |
| | PS | 45.15 |
| | OA + Phos-90G (1:1) | 4.7 |
| | Probiotic GO-01 | 5 |
| PL14/57-4 | MA | 47.6 |
| | PS | 47.6 |
| | Probiotic GO-01 | 4.8^{a} |
| PL14/57-5 | MA | 90.9 |
| | OA + Phos-90G (1:1) | 4.6 |
| | Probiotic GO-01 | 4.6 |

^{a}Of matrix, loading is 4 wt %

### Method

PGSS was carried out to achieve the encapsulation of the formulations under the following processing conditions:
Temperature: 40° C.
Pressure: 10 000 KPa
Process Time: 1 hour
Stirring: continuous with baffles

The micronisation of the formulations was assessed prior to the conducting of the additional assessments referred to hereunder and it was found that full micronisation for all the formulations was achieved. This was evidenced by the presence of no residual material in the mixing chamber and a fine powder in the product chamber.

### Example 3.2: SEM Analysis

The encapsulated microparticles were visualised using Cryo-SEM to provide an image of the probiotic flakes with different lipid formulations. The SEM images resulting from this analysis are shown in FIGS. 6(a) to 6(c).

The SEM images show that the general particle size ranged from 50-200 μm which was presumed to be agglomerated lipid particles alternatively coated GO-01 probiotic. However, the images do show that PL14/57-3 had the most robust coating followed by PL14/57-4 and then PL14/57-5.

### Example 3.2: Assessment of Stability in Simulated Intestinal Fluid

Formulations containing GO-01 encapsulated with MA/PS (PL14/57-4) and MA only were tested for stability in SIF.

The results of this assessment are shown in FIG. 7.

### Results

The results of this assessment showed that MA and MA/PS coatings offer similar protection against SIF.

### Example 4.1. Formulations of Lipid-Lipid Hybrids with PVP and Lec

Lipid-Lipid hybrids of MA/PS were prepared using the methodology of the invention and then PVP and Lec were added so that gastric resistance of the formulations could be tested. The formulations analysed are shown in Table 3, hereunder:

TABLE 3
Formulations

| Reference | Ingredients | Loading (wt %) |
|---|---|---|
| PL14/62-2 | MA | 45.6 |
| | PS | 45.6 |
| | PVP | 3.75¹ |
| | GO-01 MS | 5 |
| | Probiotic | |
| PL14/62-4 | MA | 43.75 |
| | PS | 43.75 |
| | PVP | 7.5 |
| | GO-01 MS | 5 |
| | Probiotic | |
| PL14/63-1 | MA | 43.3 |
| | PS | 43.3 |
| | PVP | 7.4² |
| | Lec | 1 |
| | GO-01 MS | 5 |
| | Probiotic | |
| PL14/63-2 | MA | 42.75 |
| | PS | 42.75 |
| | PVP | 7.5 |
| | Lec | 2 |
| | GO-01 MS | 5 |
| | Probiotic | |
| PL14/63-3 | MA | 41.85 |
| | PS | 41.85 |
| | PVP | 7.52 |
| | Lec | 3.76¹ |
| | GO-01 MS | 5 |
| | Probiotic | |
| PL14/63-4 | MA | 40 |
| | PS | 40 |
| | PVP | 7.5² |
| | Lec | 7.5² |
| | GO-01 MS | 5 |
| | Probiotic | |
| PL14/64-1 | MA | 40 |
| | PS | 40 |
| | PVP | 7.5 |
| | Lec | 7.5 |
| | GO-01 MS | 5 |
| | Probiotic | |
| PL14/64-2 | MA | 40 |
| | PS | 40 |
| | PVP | 7.5 |
| | Lec | 7.5 |
| | L. reuteri | 5 |
| PL14/64-3 | MA | 40 |
| | PS | 40 |
| | PVP | 7.5 |
| | Lec | 7.5 |
| | B. bifidum | 5 |

¹Of matrix, loading is 4 wt %
²Of matrix, loading is 8 wt %

PGSS was carried out to achieve the encapsulation of the formulations under the following processing conditions:
Temperature: 40° C.
Pressure: 10 000 KPa
Process Time: 1 hour
Stirring: continuous with baffles

### Example 4.2: Gastric Resistance Tests in SGF of Lipid-Lipid PVP Formulations

Tests were conducted on the abovementioned formulations to assess their gastric resistance after exposure to SGF for 2 hours at pH 1.2 and the results of the same are shown in FIG. 8.

### Results

As can be seen in FIG. 8 even at 8% wt PVP loading gastric protection (resistance) was maintained.

### Example 4.3: Gastric Resistance Tests in SGF of Lipid-Lipid/PVP/Lec Formulations

Tests were conducted on the abovementioned formulations to assess their gastric resistance after exposure to SGF for 2 hours at pH 1.2 and the results of the same are shown in FIG. 9.

### Results

As can be seen in FIGS. 8 and 9 it was shown that gastric resistance is maintained even with the addition of PVP and Lec to the eutectic MA/PS hybrid

### Example 5.1. Formulations of Lipid-Lipid Hybrids with PVP and Lec for Single Strain Probiotics

Single strain Lr-2 and Br-2 were encapsulated with MA/PS hybrids with 8 wt % PVP and Lec in accordance with the methodology of the second aspect of the invention and further stability assessments of the same were done as compared to unencapsulated controls of the same single strains.

### Example 5.2: Gastric Resistance Tests for Encapsulated and Unencapsulated Single Strains in SGF

The gastric resistance of encapsulated Bb-2 and Lr-2 was assessed as compared to free Bb-2 and Lr-2 in SGF and the results of this assessment are seen in FIG. 10.

### Results

Viable colonies of both unencapsulated Bb-2 and Lr-2 were eliminated after exposure to SGF whilst encapsulated Bb-2 and Lr-2 showed gastric resistance with slightly lower viability counts shown for encapsulated Bb-2 which was expected given that this probiotic strain is inherently less stable than Lr-2.

### Example 5.3: Shelf Stability Tests for Encapsulated and Unencapsulated Single Strains

The shelf stability of the same formulations as set out in Example 5.1. (and the corresponding unencapsulated controls) were tested in terms of shelf stability at 25° C. and 50% relative humidity and the results of the assessment are shown in FIG. 11.

### Results

Unencapsulated Bb-2 shows very poor stability with no viable counts being detected at any stage whilst the encapsulated Bb-2 still shows viability after 7 weeks of storage with viability of slightly above the recommended therapeutic level for viability of 106 cfu/g.

Unencapsulated Lr-2 shows stability till approximately 3 weeks whilst encapsulated Lr-2 is stable with colony counts even higher than those shown for encapsulated Bb-2 at 7 weeks.

None of the controls or strains showed acceptable stability for therapeutic efficacy by 9 weeks. However, it is clear that the shelf stability of both strains is increased dramatically when the same are encapsulated using the methodology of the second aspect of the invention.

### Example 6: Assessment of the Simulated Gastric Stability of a Probiotic that is Encapsulated Using the Encapsulation System of the Invention Versus Free Probiotic Strains

Free probiotic strains and probiotic strains encapsulated using the encapsulation system of the invention were compared to get a comparison between the survival rates of each when exposed to SGF (pH1.2) through the assessment of viability counts.

### Results

The results of this assessment are illustrated in FIG. 12 and show that none of the free probiotics survive simulated gastric (highly acidic) conditions whilst all of the encapsulated probiotics did with the survival rate of probiotic MS being the best. As such the encapsulated probiotics of the invention show much higher gastric stability during simulated gastrointestinal transit which would allow for release upon reaching the site of interest which is the intestine.

### Example 7: Comparison of the Shelf Stability of a Probiotic that is Encapsulated Using the Encapsulation System of the Invention Versus Free Probiotic Strains

Free probiotics Lr and Bb as well as encapsulated probiotics Lr and Bb were used in this assessment with respect to the shelf stability of each at 25° C. and 50% relative humidity. Stability was assessed at various time internals by assessing the number of colony forming units (cfu) at each interval over a total period of 7 weeks.

### Results

The results of this assessment are shown in FIG. 13 and indicate that free strain Bb has no viability and hence no shelf stability whilst free strain Lr initially appears to be stable but shows a rapid drop in viable colonies implying a limited shelf stability. Probiotic strains which are encapsulated within the encapsulation system of the invention show stable viable cfu counts for both probiotic Bb and Lr, which viability and therefore shelf stability is significantly higher than for the free strains.

### Example 8: Comparison of the Shelf Stability and the Gastric Survival Rate (in SGF: PH1.2) of Four Currently Available Commercial Products

Four commercial products currently available on the market and annotated Comm 1 to 4 in FIG. 14 were compared to get a comparison between the shelf stability and the survival rates of each when exposed to SGF. None of these commercial products were encapsulated.

The shelf stability was assessed by assessing the number of colony forming units (cfu) in the products as received and prior to exposure to SGF. The survival rate was assessed at various time internals by assessing the number of colony forming units (cfu) for each commercially available product after exposure to SGF.

### Results

The results of this assessment are illustrated in FIG. 14 and show that the majority of commercially available products as received (prior to simulated gastric exposure) are not stable upon receipt. This lack of stability upon receipt is indicative of poor shelf stability of these products. Albeit that Comm4 was stable upon receipt and hence may have a reasonable shelf life in comparison to the other commercially available products tested it did not survive exposure to simulated gastric conditions and hence does not show gastric stability. The gastric stability of Comm 1 to 3 could not be tested given that the same had no shelf stability and no efficacy even before gastric exposure.

Given the above experimental data the inventors can conceptualise that the encapsulation system and method for formulating the same as set out in accordance with the invention may provide a platform technology which enables the encapsulation of a variety of active compounds for use in a variety of industries and applications which is easily scalable and may therefore have significant commercial relevance. Furthermore, market entry of the invention into the nutraceuticals market should not be burdensome as no clinical trials are required prior to entry into this market which is significant in terms of both market readiness and the associated investment costs with respect to entering the market.

### REFERENCES

1. Catalano, E (2019). Chapter 3—Nanotechnology-Based Drug Delivery of Natural Compounds and Phytochemicals for the Treatment of Cancer and Other Diseases. Volume 62 (pages 91-123). https://doi.org/10.1016/B978-0-444-64185-4.00003-4
2. Nutraceuticals Market Size, Share & Trends Analysis Report By Product (Dietary Supplements, Functional Food, Functional Beverages), By Region (North America, Europe, APAC, CSA, MEA), And Segment Forecasts, 2021-2030. https://www.grandviewresearch.com/industry-analysis/nutraceuticals-market
3. Sonawane, S H and Potdar, S. B (2020) Encapsulation of Active Molecules and their Delivery System, https://www.sciencedirect.com/topics/chemical-engineering/encapsulation
4. Mazdaei, M. & Asare-Addo, K., (2022) “A mini-review of Nanocarriers in Drug Delivery Systems”, British Journal of Pharmacy 7(1). doi: https://doi.org/10.5920/bjpharm.780
5. Maria Leena, M; Mahalakshmi, L; Jeyan, A; Anandharamakrishnan, M. C (2020). Chapter 14-Nanoencapsulation of nutraceutical ingredients Biomedical and Food Applications (Pages 311-352). https://doi.org/10.1016/B978-0-12-816897-4.00014-X
6. Probiotics in Food, Beverages, Diary Supplements and Animal Feed; BCC Market reports FOD035G, January 2020.
7. Nano-microdelivery systems for oral delivery of an active ingredient. (WO2014/166994A1).
8. Microcapsulate and processes for the manufacture thereof (U.S. Pat. No. 8,637,104B2)
9. Supercritical antisolvent Micronization (High Tech Extracts) Supercritical Anti-Solvent Micronization-HIGHTECH Extracts
10. Therapeutic foods (2018): https://www.sciencedirect.com/topics/agricultural-and-biological-sciences/phytochemical#:~:text=Phytochemicals%20are%20defined%20as%20bioactive,diseases%20(Liu%2C%202004).

1. An encapsulation system, for encapsulating at least one active compound, comprising:
(a) an encapsulating material selected from the group comprising:
a eutectic mixture, a eutectoid, a hypereutectic mixture and a hypoeutectic mixture;
(b) at least one additive; and
(c) at least one active compound,
the encapsulating material and at least one additive being homogenously combined with the at least one active compound in a solvent medium comprising a supercritical compound to form a matrix which encapsulates the at least one active compound so that, in use, the at least one active compound is protected from both ambient environmental factors and gastric attack.

2. The encapsulation system, as claimed in claim 1, wherein the at least one active compound is selected from the group comprising probiotic, bacteria, yeast, vitamins, minerals, phytochemicals, phytogenics, essential oils, flavourants, biological actives and active pharmaceutical ingredients (APIs).

3. The encapsulation system, as claimed in claim 1, wherein the encapsulating material is formulated using a combination of at least two lipids, alternatively the combination of at least one lipid and one polymer; at least one lipid remaining intact upon exposure to gastric conditions in the stomach of a human and/or animal thereby ensuring that the matrix is resistant to gastric attack and therefore only solubilised by emulsification and/or lipolysis which occurs in the intestine of the human and/or animal.

4. The encapsulation system, as claimed in claim 1, wherein the encapsulating material upon combination with the at least one additive provides for stability of the at least one additive within the matrix.

5. The encapsulation system, as claimed in claim 1, wherein the additive is selected from the group comprising: hygroscopic compounds and polymeric amphiphiles or a combination of the aforementioned.

6. The encapsulation system, as claimed in claim 5, wherein the hygroscopic additive is a desiccant selected from the group comprising: natural and synthetic polymers, sugars, clay, salt and silica gel; the hygroscopic additive functioning, in use, to lower the water activity around the at least one active compound thereby protecting the same from ambient environmental humidity.

7. The encapsulation system, as claimed in claim 5, wherein the polymeric amphiphilic additive is selected from the group comprising: peptides, phospholipids, surfactants, fatty acids and glycolipids; the polymeric amphiphilic additive functioning, in use, to disrupt the crystalline structure of lipids contained in the matrix and/or influence the molecular polarity of the matrix and/or to assist in the prevention of the formation of micro-cracks in the lipid.

8. The encapsulation system, as claimed in claim 7, wherein the molecular polarity of the matrix having been influenced through the action of the polymeric amphiphile allows for the encapsulation of polar active compounds by the encapsulation system.

9. The encapsulation system, as claimed in claim 1, supercritical compound is selected from the group comprising: supercritical (sc-) carbon dioxide, water, hexane, methane and ethanol, most preferably sc-CO₂.

10. The encapsulation system, as claimed in claim 1, wherein the supercritical compound is produced utilising pressures and temperatures at or higher than the defined critical temperatures and pressure for said compound.

11. The encapsulation system, as claimed in claim 1, wherein the supercritical compound is produced in anhydrous and anoxic conditions.

12. The encapsulation system, as claimed in claim 1, wherein the encapsulating material when combined with the at least one active compound forms microparticles having a size of about 5 to about 150 μm.

13. A method of formulating the encapsulation system of claim 1 comprising:
i. producing supercritical fluid through the application of at least critical temperature and pressure to said fluid to produce a supercritical solvent;
ii. mixing the supercritical solvent with a combination of encapsulating material, at least one additive and at least one active compound so as to allow the mixture to rapidly cool upon the evaporation of the supercritical solvent
said rapid cooling providing a temperature lower than the solidification temperature of the combination of encapsulating material, at least one additive and active compound which results in the atomisation and solidification of the combination of encapsulating material, at least one additive and active compound resulting in the formation of a microencapsulated active compound or compounds.

14. A medicament produced in accordance with method of claim 13 for use in delivery of at least one active compound to a human or animal in need thereof.

15. The medicament, as claimed in claim 14, wherein the medicament is indicated in the treatment of diseases and/or for use as a nutraceutical.

16. A method of treatment of the human and/or animal body by administering the medicament of claim 15 to a human and/or animal in need thereof.

17. The encapsulation system, as claimed in claim 1, being indicated for application in the agricultural industry for the encapsulation of biopesticides, alternatively feed additives, further alternatively growth factors.
